(19) United States
(12) Patent Application Publication
(10) Pub. No.: US 2021/0317539 A1
(43) Pub. Date: Oct. 14, 2021

(54) METHOD FOR THE QUALITY CONTROL OF SEED LOTS

(71) Applicant: LIMAGRAIN EUROPE, Saint-Beauzire (FR)

(72) Inventors: Nathalie RIVIERE, Orcet (FR); Jordi COMADRAN, Riom (FR); Sandra CONTAMINE, Nebouzat (FR); Jean-Pierre MARTINANT, Vertaizon (FR); Guillaume COLLANGE, Aubiere (FR); Aurélien AUDES, Billom (FR)

(21) Appl. No.: 17/264,427

(22) PCT Filed: Jul. 29, 2019
(86) PCT No.: PCT/EP2019/070386

§ 371 Date: Jan. 29, 2021

(30) Foreign Application Priority Data
Jul. 30, 2018 (FR) 1857115

(51) Int. Cl. C12Q 1/6895; C12Q 1/6806

(52) U.S. Cl. CPC C12Q 1/6895; C12Q 2600/142; C12Q 1/6806

(57) ABSTRACT

The invention relates to a method for the quality control of the varietal purity of seed lots by analysing sub-lots of the seeds, said control being carried out by sequencing the genes of interest.

The invention relates to a quality control process in the field of seeds and varietal purity.

The marketing of seeds is subject to the control of their purity rate. This rate is specific to each species but must be 98% by weight or more (Directive 66/402/EEC on the marketing of cereal seed), this standard also applies to seeds which are marketed for the production of basic seeds, pre-basic seeds, the production of certified seeds or the production of hybrids. This varietal purity is mainly checked by field inspection, in the case of hybrid seed production with a male sterile parent, the purity level of this parent must be even higher (99.9% for maize).

The availability of an alternative quality control solution to field inspection is of interest to seed companies, especially by the need to have a rapid evaluation, without waiting for the plant development necessary for phenotypic evaluation.

Moreover for these companies, the control of varietal purity is not limited to the steps mentioned above, each step upstream of the basic seed production is concerned by this requirement of varietal purity. It is reminded that the varietal purity rate is defined as the percentage of plants coming from a lot and which are in conformity with the description of the variety. This percentage is expressed in weight of seeds.

In hybrid seed production, the improvement of the quality of agricultural seed production is achieved by verifying the genetic purity of the basic seed lots (parental lines used for hybrid production) used in commercial seed production. This purity is assessed by the detection and identification of contaminating seeds in a sample batch of the parental seeds.

Contaminants are seeds of the same species, but with genetic variations at some loci in their genome, relative to the genotype expected for the seeds of the lot under consideration. In the seed lot production process, the presence of contaminants is reduced through vigilance in the upstream production steps, cultivation practices, purification, isolation, and controls performed throughout the process. Thus, almost all the seeds in the lot have the same genotype, the contaminants being present at a generally low percentage and indeed the level tolerated in a lot for it to be marketed must be less than 2%.

The identification of genetic traits of interest is also important in seed commercialization, indeed some traits ensuring for example tolerance to a herbicide or a pathogen (for example Late Blight in Sunflower) bring a certain added value to a seed lot and when a variety is commercialized as a carrier of this trait, a verification of the presence of this trait in the seed lot will be interesting. By trait it is meant the allelic form of a locus linked to a phenotypic trait.

A similar problem concerns the adventitious presence of CMOs or any other alteration in the genome. The commercialization of non-GMO plants requires proof of the absence of CMOs or the presence of a rate lower than a percentage determined by the regulations. In contrast, the regulations in some countries, for certain GMO traits, such as insect resistance, require that seeds containing the GMO are sold with a certain percentage of seeds not possessing the GMO trait, in order to provide refuge zones for the insect.

The massive development of SNP (Single Nucleotide Polymorphism) markers and high-throughput genotyping technologies has led to the development of marker-assisted breeding. Genotyping is typically performed using different technologies, either by PCR (Kasp—LGC Genomics, Taqman—Life Technologies) or hybridization on DNA chips (Axiom—Life Technologies, Infinium—Illumina).

If the Taqman quantitative PCR technology is today considered as the reference for the detection of adventitious presence of GMO plants in a mixture of non-GMO plants, it is based on the detection of a presence/absence type polymorphism of a given sequence, and not on a polymorphism between different allelic forms of a SNP. Thus, in this particular case of GMO detection, the polymorphism relates to the presence of a trait that can be amplified (amplicon) and therefore easily identifiable.

The estimation of the purity of seed lots, understood as the absence of GMO trait, has been studied by Remund (Seed Science Research (2001) 11, 101-119), two solutions have been identified by these authors to limit the resources necessary for these verifications and in particular the analysis in pool. They indicate that this method is effective when one is looking for the absence of a particular individual, on the other hand when a purity level is sought it is preferable to work seed by seed. These authors have developed a tool Seedcalc, which allows a quantitative approach by adjusting the number of pools and the number of seeds per batch, this method is particularly suitable for real-time PCR (Laffont, Seed Science Research (2005) 15, 197-204).

However, an example of using seed pools to check varietal purity exists. The application WO 2015/110472 proposes to analyze seed lots by manual or semi-automatic sampling of a determined sample volume from one or more seeds, this volume being determined to allow the analysis of at least one constituent of the seed(s). Tissue taken from several seeds is placed in an identified and traceable well and the analysis of the said constituent is then performed on the contents of the well(s). This method of bulk constitution makes it possible to make varietal purity (example 6). This purity is evaluated by the Kaspar method (KBioscience) from bulks of 5 and 10 seeds, the presence of a contaminant in these bulks is characterized by the presence of a heterozygous cluster, however the authors indicate that this cluster is close to the homozygous cluster and that it is easier to identify for a bulk of 5 seeds than for a bulk of 10 seeds.

The development of high-throughput sequencing technologies, or NGS (Next Generation Sequencing) has revolutionized the world of genomics, allowing the massive discovery of SNP markers between lines of a given species. These techniques allow a large number of possible sequence readings in a single experiment.

Sequencing depth allows the identification of a weakly represented allele when identifying allelic forms for a pool of individuals. It can also be used to identify a number of allelic forms greater than two for the same locus. Thus, the sequencing of amplicons allows the targeted study of loci of interest, the identification of SNPs and the characterization of the allelic composition of an individual or a mixture of individuals. An application in research is the detection of rare mutations in a mutagenized population (TILLING, Targeting Induced Local Lesions in Genomes). In these applications the identification of rare alleles in pools can be combined with 2D or 3D pools of individuals allowing a reduction in the number of pools to be analyzed (Tsai et al, Plant Physiol. 2011 July; 156(3):1257-68; Taheri et al, Mol Breeding (2017) 37:40; Gupta et al, The Plant Journal (2017) 92, 495-508) WO2014134729, EP 2 200 424). This approach can also be applied to the identification of mutations by Gene Editing methods (Kumar et al, Mol Breeding (2017) 37:14). However, these approaches remain qualitative, there is no quantitative consideration.

The possibility of using pooled sequencing genotyping has been evaluated for the identification of allelic frequencies in populations by Gautier (Mol Ecol. 2013 July; 22(14):3766-79). However, this approach is particularly well suited to the analysis of large populations over a large number of SNPs, and does not seem to be suitable for the detection of rare alleles (generally less than 5%).

One of the difficulties in finding a rare allele is the reliability of the result, as the frequency of the rare allele is close to the sequencing error rate.

In the case of quality control of seed lots, the goal is to detect the presence of a contaminant, to accurately estimate the rate of the contaminant within the seed lot from which the analyzed sample originated, and preferably to determine its genetic profile to better understand its origin. Detection can be carried out by the analysis of loci of interest, chosen by the skilled person, according to his knowledge of the genetic material to be qualified and of the genetic material likely to contaminate it.

Thus, Chen et al (2016, PLOS ONE 11(6)) have developed, for maize, two sets of SNPs for quality control: a set of markers for rapid control, using a reduced number of SNPs (50-100) to identify potential errors in the labelling of seed packages or plots, and a larger set of markers, used for finer characterization and discrimination of genetic material. In this example, sampling 192 individually analyzed individuals would give a probability close to 100% to detect a 5% contamination in a lot, but this probability becomes less than 90% for a 1% contamination.

In the case of quality control of basic seed lots, the expected genetic purity is high, as well as the desired precision of estimation, which depends both on the number of seeds sampled (tested) and the number of seeds in the basic seed lot. For example, if 200 seeds are tested and the impurity level is 0%, the confidence interval of this value ranges from 0% to 1.49%. Therefore, the number analyzed is too small to guarantee a sufficient level of purity by analyzing only 200 grains. On the other hand, when analyzing 2000 grains, a 0% impurity level has a confidence interval of 0% to 0.15%. However, even if genotyping costs have been considerably reduced, such sampling, combined with plant-to-plant treatment, is not economically viable for quality control.

Genia (Montevideo, Uruguay) offers a method for determining genetic purity in batches of lines and identifying contaminants by analyzing a unique mixture of 10,000 seeds and sequencing amplicons targeting approximately 350 SNPs. This company claims to determine varietal purity with a sensitivity of 0.8% and a confidence interval of 99%. This approach is similar to that developed by Gautier et al. in that it is based on a statistical model for estimating allele frequencies on a large number (350) of SNPs, from which an estimate is made of the frequency of the different genetic profiles present in the mixture. However, such an approach does not reliably detect a rare allele for a given SNP, which is necessary in the search for contamination for a given trait.

It is therefore necessary to have a cost effective method, allowing the analysis of a large number of individuals, in order to accurately determine the genetic purity of a given seed lot, especially for seed lots with a high level of purity.

The method presented here is based on the estimation of the purity of a seed lot based on the binary qualitative analysis (presence/absence of a contaminant) of several sub-lots of samples. The analysis on each sub-lot consists in detecting the presence of an alternative allele at one or more loci of interest by sequencing amplicons. The number of sub-lots, as well as the size of each sub-lot are defined according to the expected purity level (estimated by the operator) and the precision sought, and in such a way that there is preferably a statistical probability of finding a maximum of one contaminant in a given sub-lot. This means that, from a given number of seeds used for the test, at least as many sub-lots as the estimated number of contaminants are formed, preferably exactly as many sub-lots as the estimated number of contaminants. Furthermore, because of the analysis of several sub-lots, the method makes it possible to distinguish between contamination by a hybrid (segregation) and contamination by a lineage (no segregation), by comparing the contaminant profiles of the different sub-lots.

However this method is not limited to this binary approach, indeed the use of sequencing makes it possible not to limit the method to the identification of two allelic forms and in this context the method also allows identification of contaminants in seed lots heterozygous for the considered allele, the contaminant being heterologous to the allelic forms of this individual.

The invention thus relates to a method for determining the quantity of contaminants at at least one locus of interest, present in a seed lot of a variety of interest, characterized in that
(a) seeds from a seed lot are grouped into sub-lots of at least 10 seeds, the number of sub-lots so obtained being greater than or equal to 10
(b) targeted sequencing of at least the region of the seed genome containing the locus of interest is performed for each sub-lot,
(c) the presence of a contaminant is qualitatively determined for each sub-lot if an alternative allele to the expected allele(s) is detected (there may be several expected alleles at a single locus, in particular if the seed is seed of a hybrid plant) for each sequenced genomic region (presence/absence of an alternative allele)
(d) the quantity of contaminants in the overall lot is determined by compiling the qualitative results obtained for all sub-lots.

Optionally and preferentially, and in order to perform sequencing, the region corresponding to the locus of interest between step a) and step b) is amplified by PCR. This amplification step is performed directly on all seeds in each sub-lot. Alternatively, the sequencing of step b) is performed on the DNA extracted from the seeds present in a sub-lot, the region of the seed genome containing the locus of interest being optionally amplified. In another embodiment, the RNA present in the seed lot is also extracted, reverse transcription is performed to obtain complementary DNA (cDNA), and optionally an amplification of the loci of interest of this cDNA, and the sequencing of loci of interest (preferably amplified) is also performed on the obtained cDNA.

The estimation of the impurity \hat{p} of the batch is obtained according to the formula:

$$\hat{p} = 1 - {( 1 - \frac{d}{n} )}^{\frac{1}{m}}$$

in which n is the number of pools; m is the number of grains in a pool; d is the number of pools in which a contaminant has been identified.

This is the formula proposed by Remund (2001, op. cit.), which notably allows to take into account the fact that contaminant investigations are carried out only on a sample of the seed lot and thus to take into account the biases potentially induced by this sampling.

This process thus makes it possible to calculate the percentage of contaminants in the seed lot (and thus the purity of the seed lot: 1−\hat{p}).

A contaminant is a seed with an allele different from the expected allele at the locus of interest given in that seed batch. However, when the method is implemented on several loci of interest, it may be decided that a lot is contaminated only when unexpected alleles are observed at more than one locus in that lot, e.g. at 2 or 3 loci.

Preferably, in step a), a maximum number of seeds is used, calculated so that at most one contaminant is statistically present in each seed sample (sub-lot). In industrial production methods, a purity level of more than 99% is generally observed. Thus, with a count of about 100 seeds, for example between 80 and 120, one can expect to detect predominantly one contaminating seed. The methods described above are indeed implemented for homogeneous seed lots, i.e. for which at least 95%, preferably at least 96%, more preferred at least 97%, more preferred at least 98%, more preferred at least 99% of the seeds have the same genotype. Depending on the estimated purity of the seed lot, sub-lots contain a maximum of 20, or a maximum of 50, or a maximum of 80, or a maximum of 100, or a maximum of 200, or 2000 seeds. When assessing a characteristic for which the expected purity is of the order of at least 90%, respectively at least 95% (such as the germinative character of the seeds), the quantity of seed in each sub-lot prepared in step a) is then of the order of 10, respectively 20, i.e. between 15 and 25.

Step b) of the process consists in the targeted sequencing of at least one genomic region, containing the locus of interest for which the presence of a contaminant is sought.

It is clear that this sequencing step is performed on nucleic acid. Therefore, the DNA of the batches is prepared, for example by crushing the seeds and using the flour or isolating the DNA from the flour. These methods are known in art. As seen above, cDNA can also be prepared.

This sequencing step is preferably performed by high throughput sequencing (HTS). Different technologies (Illumine®, Roche 454, Ion torrent: Proton/PGM (ThermoFisher) or SOLID (Applied BioSystems)).

In summary, these HTS technologies have 2 steps in common:
an amplification step, by PCR
a sequencing step, this step being carried out by different approaches depending on the technology used.

The Illumine® technology uses clonal amplification and synthetic sequencing (SBS). A double-stranded DNA library is generated from the sample to be analyzed by PCR amplification and addition of specific adapters at the ends, then the DNA is denatured into single strands, and the ends of the single strands are randomly attached to the surface of the flowcell, on which a solid phase bridge PCR is performed (creation of dense clusters where the fragments are amplified).

Sequencing is performed by adding the 4 labeled reversible terminators, primers and DNA polymerase, then the fluorescence emitted by each cluster is read, allowing the determination of the first base. Several cycles are then performed to read the whole sequence.

For the implementation of the 454 technology, a single-stranded DNA template library is obtained, with specific adapters being added at the 3′ and 5′ ends, and each DNA strand being immobilized on a bead (one DNA fragment=one bead). These beads are then integrated with the amplification products in a water-oil emulsion to create “microreactors” (each drop of water in the oil) containing a single bead. The PCR is performed in this emulsion with the whole bank being amplified in parallel, allowing to obtain several million copies per bead.

Then the beads are purified and the fragments are loaded on plates such that the diameter of the wells allows only one bead to enter at a time. Sequencing enzymes are added and the individual labeled nucleotides are sent one after the other. Sequence detection is performed by a CCD camera based on the luminescent signal.

For the SOLID technology, the banks are prepared, adapters are added and a PCR is performed in an emulsion, as in the 454 method. The amplified beads are then enriched, the 3′ end of the DNA is modified to allow a covalent fixation on a slide, and the beads are deposited on the slide. Sequencing is performed by ligation: primers hybridize to the adapters present on the matrix. A set of 4 fluorescently labeled 2-base probes are associated with the primers. The specificity of the 2-base probes is performed with the 1st and 2nd bases of each ligation reaction. Several cycles of ligation, detection and cleavage are performed. In this process each base is detected by two independent ligation reactions using two different primers. The coding system of the reading on two bases allows a very high fidelity of the reading of the results. This method makes it possible to differentiate between sequencing errors and real variants (SNPs, insertions and deletions).

For the IonTorrent technology, banks are prepared and adapters are added. Emulsion PCR is performed. Sequencing is not based on the detection of fluorescence of nucleotides or their polymerization residues by a CCD optical sensor, but uses a CMOS sensor that detects the H+ ions released during DNA polymerization. The CMOS sensor measures the pH in each of the wells, which indicates the presence of one or more bases that have been incorporated into the DNA being analyzed. The bases are added one after the other to detect which one has been integrated and then rinsed and the method is repeated.

Other sequence technologies exist such as the MinION technique from Oxford Nanopore technologies (https://nanoporetech.com/products#minion, Mikheyev and Tin (2014). Molecular Ecology Resources. 14(6):1097-102.) or Pac Bio from Pacific bioscience (https://www.pacb.com/products-and-services/pacbio-systems/).

The process described herein makes it possible to limit the risk of detecting a false positive (one mistakenly concludes to the presence of the alternative allele) or a false negative (one mistakenly concludes to the absence of the alternative allele) that these methods of NGS sequencing can present because of the sequencing error rate inherent to each technology. Indeed, step c) consists in determining the absence or presence, for a sample, of an unexpected sequence in the sequencing products. In case of presence of such an unexpected sequence (corresponding to the presence of a contaminant), there is no need to quantify the quantity of unexpected sequence compared to the quantity of expected sequence (corresponding to the correct sequence of the seeds in the seed lot). The detection is therefore only qualitative (i.e. binary: presence/absence of a sequence of an alternative allele(s) to the expected allele(s). The use of seed sub-lots also makes it possible to increase the number of seeds studied for each sequencing reaction and thus to have a sufficient sample of seeds while keeping costs under control.

The presence of such a sequence of an alternative allele is indicative of the presence of a contaminant for that allele.

This analysis is carried out for each genomic region analyzed, i.e. for each locus of interest previously determined by the person of skill in the art, and allowing to characterize the seed lot.

In fact, when the number of seeds in each sub-lot is chosen so that only one contaminant is present (statistically) within this sub-lot, the presence of an alternative allele is sufficient to conclude to the presence of a single contaminant.

The next step in the process is the calculation of the actual percentage of contaminants in the seed lot. This is done by compiling the qualitative results obtained for all sub-lots.

The purity level of the seed lot is then estimated by considering the number of contaminated sub-lots, the total number of sub-lots analyzed, and the number of each sub-lot.

The estimation of the impurity of the batch is obtained according to the formula:

$$\hat{p} = 1 - {( 1 - \frac{d}{n} )}^{\frac{1}{m}}$$

in which n is the number of pools; m is the number of grains in a pool; d is the number of pools in which a contaminant has been identified.

The confidence interval of this estimation can also be determined by any appropriate statistical method, including an F distribution, as applied in the SeedCal tool used in the framework of the ISTA (International Seed Test Association) and as explained in Remund (2001).

$${\hat{p}}_{UL} = 1 - {( 1 - \frac{( d + 1 ) {F}_{1 - α , 2 d + 2 , 2 n - 2 d}}{( n - d ) + ( d + 1 ) {F}_{1 - α , 2 d + 2 , 2 n - 2 d}} )}^{\frac{1}{m}} .$$

In a preferred mode of execution, step b) involves the targeted sequencing of several regions of the genome containing several loci of interest. This allows to better guarantee the identity of the seeds present in each sample and to detect, in a finer way, the presence of contaminants.

Thus, one can sequence in a targeted manner, at least 2, preferably at least 5, preferably at least 10, more preferably at least 100, 50, 40, 15 loci of interest, or even at least 20 loci of interest. Although there is no upper limit to the number of loci of interest that can be assessed, it is preferred to limit the number of loci of interest. Indeed, it is possible to characterize a variety with a limited number of (loci-specific) markers (between 15 and 20), and to use this set of markers to discriminate plants of this variety from other plants. A variety is understood as a set of plants with the same genetic background, the variety can be a commercialized variety, but also a line not yet registered in the catalog, a basic line, a pre-basic line or a line in the course of propagation.

The optimal number of loci of interest is defined by the person of skill in the art, according to the plant material considered, but also by setting the minimum number of loci discriminating any given pair of varieties. Thus, the minimum number of loci discriminating any pair of varieties can be set at three, limiting the risk of confusing a real contamination with an experimental false positive. Different algorithms are described by Rosenberg et al (Journal of Computational Biology 12 (9), 2005, 1183-1201) to select a set of discriminant markers.

These algorithms can be improved or modified to take into account other criteria such as the quality of the selected markers (quality refers to their ability to be amplified, unequivocally identified). Groups or categories of markers can be identified and define a subgroup of markers that preferentially contains markers from a given group or from different groups. In this way, it is possible to define a set of markers that one wishes to use.

The algorithm can also take into account the statistical quality of these markers defined as the minimum number of discriminating markers to declare a pair of individuals as different. From this criterion, the discrimination quality of a set of markers can be evaluated by the number of pairs of individuals that this set is able to discriminate, ideally the totality of individuals managed by the producer.

In the context of the present invention, the method shall preferably be implemented on loci of interest allowing both to discriminate the variety of interest (to ensure the consistency and concordance of the genetic background between plants) and to identify the presence or absence of other loci of interest (in particular related to traits of interest).

In this embodiment, i.e. when performing a sequencing of several regions of the genome, one can decide to consider that a contaminant is present in a batch only if one observes the presence of unexpected sequences for more than one locus of interest in this batch. In other words, it can be decided that, if a single alternative allele (an unexpected sequence for a single region of the genome, while the sequences obtained for the other regions are those expected) is observed in a given batch, the presence of a contaminant is not considered to be proven.

The method herein described therefore makes it possible to determine the presence of contaminants in a seed lot, in particular to control varietal purity during an industrial production process.

This method can also be performed in order to check the purity level of a trait that is sought in the homozygous state in the seed lot. In this method, only the region of the genome containing the specific trait to be monitored is preferentially evaluated. Several traits can be monitored simultaneously, using specific markers for each trait.

A trait is understood as an allelic form specific to a given locus, in this context this allelic form can be native, linked to a mutation identified by Tilling or Ecotilling, mutation linked to the imprinting of a transposable element, mutation obtained by Gene Editing or by any other method. In this context the mutation whether it is a point mutation, an insertion or a deletion involves a limited number of bases. This method can also be applied to a heterozygous trait, the contaminant will then correspond to an alternative form to the allelic forms expected in this individual.

In a preferred embodiment, a trait (which can be linked to a single allele or to several alleles) provides the plant with a phenotypic trait of interest (such as drought resistance, resistance to biotic stress, resistance to nitrogen deficiency, yield increase . . . ).

When the trait is linked to a mutation involving a large insertion, such as a GMO trait, a mutant obtained by insertion of a transposable element or a mutant obtained by Gene Editing, the method can be implemented by looking for the presence of the allelic form not containing the insertion or mutation considered. The presence of this allelic form indicates that the presence of the trait related to the mutation in a homozygous form in the seed lot is not fully guaranteed. This method can be used for example when the mutation corresponds to the introgression of a DNA fragment from another species, this specific situation will be encountered for example to check the purity of fertility restoring lines in rapeseed.

This method also makes it possible to search for the fortuitous presence of a trait, the trait whose fortuitous presence will be searched for could be a GMO, a mutation linked to Gene Editing or the introgression of a fragment coming from a heterologous species, this search will be done by amplification then sequencing of a specific region of the T-DNA, or of the insertion. By extension, this method can be applied to small mutation-related traits if primers that specifically amplify the region in the presence of the mutated allelic form can be defined. By adapting the protocol, number of batches and number of seeds per batch, the protocol can be extended to identify the presence of traits for frequencies up to 10% and in this context we can verify for example the presence of 10% of wild seeds in a batch of GMO seeds (legislation on safe areas). These applications are not limited to CMOs, the trait followed by this method can be the introgression in a lineage of a fragment from another species, the presence of a fertility restoring locus from radish in rapeseed for example. In the same way, the verification will allow to verify that this introgression is in a homozygous state.

Alternatively, the method can be used to detect the adventitious (undesired) presence of CMOs or other mutation linked to the insertion of a fragment of significant size in a seed lot. This mutation can be linked to the presence of a transposable element or to an insertion obtained in particular by Gene Editing. In this mode of realization, specific primers of a particular transgene or insertion (if a particular contamination is suspected) or different generic primers will be used to detect different transgenes without a priori.

In the case of varietal purity, markers related to these traits can also be added to the list of markers used to characterize the variety.

Thus, in a preferred embodiment, steps b), c) and d) are performed for several regions of the genome containing several loci of interest.

In this embodiment, it is preferred when a subset of several loci makes it possible to discriminate or identify a variety of interest. As seen above, this number of loci is variable and these loci can be determined by one of skill in the art, in particular according to the teachings of Rosenberg (cited above). In a particular mode of the invention, he will be able to include information concerning the production plan, involving particular controls and measures: isolation distances, border zones, castration, which implies that the risk of contamination will be limited and the seed lot will a priori be uncontaminated or weakly contaminated. Furthermore, due to these measures, a contamination will most likely come from a known contaminant, notably from a parental line, including parental lines involved in the production of basic and pre-basic seeds. In this particular context the number of markers to identify the purity of a line may be very small, in particular 20 or less.

As seen above, in one embodiment, a lot is declared as containing a contaminant if an alternative allele to the expected allele is observed for a single locus of interest. In another embodiment, a batch is declared as containing a contaminant if an alternative allele to the expected allele is observed for more than one locus of interest (in particular 2 or 3 loci).

In one embodiment, at least or exactly one locus of interest is linked to a character of interest (trait). In another embodiment, it is a combination of loci that is linked to a character of interest (trait).

In one embodiment, at least one locus of interest is linked to a specific trait a priori not present in the seeds of the lot. In this embodiment, one looks for the fortuitous presence of this trait. Markers are therefore added to check the absence of the trait. In this embodiment, the method is essentially qualitative. The integration of these markers in the claimed protocol makes it possible to carry out in a single experiment additional controls necessary elsewhere.

In general, a lot is considered to be non-compliant if the frequency of the unwanted trait(s) is higher than 10% in the seed lot.

In a preferred mode of production, the quantity of seed in each sub-lot prepared in step a) is between 80 and 120.

The method herein described can also be used to determine intrinsic agronomic characteristics of the seeds present in the lot. Hence, one can determine the expression of genes that will lead to undesired seed properties (e.g. dormancy marker genes which, if expressed, are a marker of seed non-germination). In order to determine the expression of these genes in the seeds of the lot, RNA is extracted and reverse transcription is performed. Thus, the process described above may also include the following steps:
i) RNA is further extracted from the seeds of the sub-lot and reverse transcribed into cDNA before step b).
ii) sequencing of this cDNA using primers specific for dormancy genes is carried out at the same time as the sequencing of step b) is carried out
iii) the presence of non-germinative seeds is determined qualitatively for each sub-lot, if cDNA relating to dormancy genes is detected in sequencing step (ii) (presence/absence of cDNA)
iv) the amount of dormant seeds in the overall lot is determined by compiling the qualitative results obtained for all sub-lots in (iii).

Steps iii) and iv) are carried out in the same way as described above. Seeds in the lot generally do not exhibit the dormancy trait and by appropriately selecting the number of seeds in the sub-lots, the qualitative information from iii) can be used to obtain quantitative information. For example, if it is known that no more than 5% of the seeds exhibit the dormancy characteristic (a situation generally observed in commercial seed lots, where at least 95% of the seeds properly germinate), sub-lots containing in the order of 20 seeds (between 15 and 25 seeds) are used.

This dormancy problem is particularly important for seeds of sunflower, wheat, rice.

Dormancy marker genes whose expression is evaluated by sequencing the cDNA obtained from seed RNA are preferentially selected from genes known in the art, some of which are described below.

In another embodiment, a trait can correspond to a level of expression of a marker gene. For example, the germinative quality of a seed lot is an essential characteristic, and this quality may change during seed storage.

A state in which a seed does not germinate when it is in favorable germination conditions (temperature and humidity) is named a dormancy state. Dormancy reflects an adaptation of plant species to environmental conditions (ability to put itself in a latent state in the absence of favorable conditions for plant development). Thus sunflower, rice or sorghum show a dormancy whose removal is accompanied by an improvement in germination at low temperatures, while in the case of wheat, barley or oats, it is an improvement in germination at higher temperatures (Baskin and Baskin, Seed Science Research (2004) 14, 1-16).

This property is particularly important for cultivated species, the objective being to produce and market seed lots with the ability to germinate quickly and homogeneously after sowing. It is therefore important to be able to characterize the level of dormancy of a seed lot, and such analyses are routinely performed in factories, through germination tests, these tests use in particular Ethrel which has the ability to remove the dormancy. However, these analyses are long and labor-intensive, hence the interest of being able to replace them with molecular analyses.

Studies performed in different species have identified genes whose level of expression correlates with the dormancy or non-dormancy state of the seeds. Bessel et al (PNAS Jun. 7, 2011 108 (23) 9709-9714; Trends in Plant Science, June 2016, Vol. 21, No. 6, 498-505) identified sets of genes co-expressed specifically according to the state of dormancy or non-dormancy in Arabidopsis thaliana. For example, the DOG1 (Delay Of Germination 1) gene is involved in maintaining dormancy at low temperatures in Arabidopsis, and the role of this gene appears to be conserved between species such as in lettuce (Huo et al., PNAS Apr. 12, 2016 113 (15) E2199-E2206) or wheat (Ashikawa et al., Transgenic Res (2014) 23: 621). In sunflower, Layat et al. (New Phytologist (2014) 204: 864-872) analyzed the RNA abundance associated with the polysomal fraction in dormant and non-dormant embryos, and identified genes associated with the dormancy state, such as HSP (HSP70, HSP101) and stress response genes or involved in the signaling pathways of abscisic acid (ABA), a hormone associated with the maintenance of dormancy. Conversely, other genes, such as alpha tubulin, are specifically expressed in non-dormant seeds (Layat et al., op. cit).

Thus, the analysis of the expression of a gene specific to the dormancy state makes it possible to characterize the germinative quality of a batch of seeds. The objective being to qualify lots for their germination capacity, the analysis of the expression of a specific gene of the dormancy state allows to determine the percentage of dormant seeds in a non-dormant lot, by semi-quantitative analysis. In the case of a high dormancy rate, in particular >1%, the joint analysis of a gene specific for the dormant state and a gene specific for the non-dormant state would allow, by calculating the relative abundances of these two genes, to express a dormancy rate. Similarly, other evaluations of the physiological status of the seeds could be carried out, thus replacing tests carried out in the laboratory. The appropriate marker gene can be selected based on the timing of this phase of sequencing testing. These tests can be performed, for example, shortly before packaging the seeds for commercialization. This evaluation will include the quality of priming, germination ability, vigor and viability of the seeds. The germination ability is described in particular in application WO 2018/015495.

The method described above may also be used to determine the specific purity of the seed lot, i.e. the presence or absence (and quantification) of seed from a species other than the species of the seed in the seed lot. Such analysis is currently routinely performed by operators, who visually determine the presence or absence of seeds of unwanted species (ISTA (International Seed Testing Association) rules chapter 4).

A process as described above can therefore be implemented, characterized in that
i) DNA sequencing of the sub-lots is also carried out using primers specific to one or more species different from those of the seeds in the sub-lot, at the same time as the sequencing in step (b) is carried out.
ii) the presence of seeds of different species is determined qualitatively for each sub-lot, in case of detection of genes belonging to said species (presence/absence of genes specific to other species)
iii) the quantity of exogenous seeds in the overall lot is determined by compiling the qualitative results obtained for all sub-lots in ii).

In this method, the presence of weed as a different species is sought in particular. In particular, the presence of seeds of Aeginetia, Alectra, Orobanche and Striga is sought. The presence of sclerotia will also be routinely searched for.

Steps ii) and iii) are carried out in the same way as described above. Seeds in the lot generally do not have many seeds of other species and, by adequately selecting the number of seeds in the above lots, the qualitative information in iii) can be used to obtain quantitative information. For example, if it is known that no more than 1% of the seeds present are from a species other than the species of interest, (which is usually the case in commercial seed lots, where at least 99% of the seeds are of the species of interest), sub-lots of the order of 100 seeds (between 80 and 120 seeds) are used.

The method described above can also be used to detect the presence of pathogens in the seed lot (contamination) (see ISTA (International Seed Testing Association) rules chapter 7). For example, the quantity of Botrytis contaminated sunflower seeds tolerated for the marketing of a sunflower seed lot is 5%.

A process as described above can also be implemented by carrying out the following steps in addition:
i) Sequencing of the DNA or cDNA contained in the sub-lots using primers specific to pathogenic species is carried out at the same time as the sequencing of step b) is carried out.
ii) the presence or absence of DNA of the pathogenic species is determined for each sub-lot if sequences belonging to those pathogenic species are detected
iii) the conclusion as to the contamination of the lot is based on the presence of sequences belonging to the said pathogenic species.

A gene from any pathogen, such as a bacterium, fungus, virus or insect can be sequenced. This method is particularly suitable for detecting the presence of Xanthomonas campestris pv. campestris in Brassica ISTA seeds (rules 7-019a: Detection of Xanthomonas campestris pv. campestris in Brassica spp. Seed) or Berg (Plant Pathology (2005) 54, 416-427). A PCR test for the identification of a pathogen on seed exists for the identification of downy mildew on sunflower (loos et al., Plant Pathology (2007) 56, 209-218). It has the advantage of detecting a pathogen on seed, whereas the presence of this pathogen on the seed does not cause symptoms, especially at the very low levels sought. This protocol indicates primers, the fact of making a sequencing and not a revelation on gel will allow to have a better precision. The identification of Clavibacter michiganensis on tomato can also be performed (Hadas et al, Plant Pathology (2005) 54, 643-649).

In order to implement the processes described above, the following steps can be carried out before step b).
i) DNA is extracted from each sub-lot of seeds.
ii) RNA is extracted from each seed sub-lot and reverse transcribed into cDNA.
iii) The DNA extracted in i) and the cDNA obtained in ii) are mixed.
iv) Optionally, amplification is carried out on the DNA obtained in iii), specific to certain loci, or non-specific amplification.
v) The DNA obtained in iii) or the amplification products obtained in iv) is used as template for the sequencing step.

In one embodiment, steps i) and ii) can be carried out simultaneously, the extraction of DNA and RNA can be carried out in particular using Macherey-Nagel's total DNA, RNA and protein isolation NucleoSpin® TriPrep kit.

Thus, in a preferred embodiment, step iv) is carried out by amplifying specific sequences of genes (in particular from other organisms) whose absence or presence is wished be to verified. The aim is to determine whether these other organisms are present in quantities below the tolerated levels for commercialization. in particular, the presence of viral sequences can thus be detected. A non-specific amplification of the entire DNA of the genome can also be performed.

In another embodiment, step iv) can also be carried out by amplifying specific sequences allowing the determination of certain agronomic properties of the seeds of the sub-lot, at least one agronomic property of the seeds being chosen among the state of dormancy, in particular the quality of priming, the aptitude for germination, the vigor and the viability of the seeds.

In an embodiment, the process contains the steps:
i) in addition to the isolation of the DNA, an extraction of RNA from the seeds of the sub-lot, and a reverse transcription of this RNA into cDNA is also carried out before step b)
ii) sequencing of this cDNA is performed using primers specific to genes related to an agronomic property of the seeds, at the same time as the sequencing of step b) is performed
iii) the presence of seeds with the agronomic property is qualitatively determined for each sub-lot, in case of detection of cDNA relating to the specific genes of the agronomic property of the seeds in the sequencing step (ii) (presence/absence of cDNA)
iv) the quantity of seeds with this agronomic characteristic in the overall lot is determined by compiling the qualitative results obtained for all sub-lots in (iii).

Generally, the agronomic property of the seed is selected from the dormancy state, including priming quality, germination ability, vigor and viability of the seed. Several agronomic properties can also be sought by sequencing suitable genes.

The marker gene for the physiological state and the agronomic property of the seeds is selected among the genes that are expressed, in the seeds, at the same time as the unwanted agronomic character, (dormancy, lack of vigor . . . ). Thus, an absence of expression of this gene is desired and it is generally desired that the expression of this gene is not present in more than 10% of the seeds of the seed lot.

In a preferred embodiment, and in the implementation of varietal purity analysis (do the seeds present contaminants (i.e. undesired alleles) at loci of interest), one can identify the contaminant(s) present in the seed lot.

For each sub-sample, a molecular profile can be defined corresponding to the compilation of data for each locus of interest. The profile of each sub-sample can then be compared to the expected molecular profile, and a contaminant molecular profile can be deduced by subtraction. Thus, a locus of interest with no alternative allele will be considered identical to the locus between the expected variety and the contaminant, while a locus with an alternative allele will be defined as potentially homozygous for the alternative allele, or heterozygous as expected allele/alternative allele.

These contaminant molecular profiles can then be compared to a reference database in order to identify the nature of the contaminant, and possibly the moment it entered the production cycle.

Thus, a contaminant identification process is envisaged, which implements the method as described above, and which also includes the steps of
i) defining the molecular profile of the contaminant in each contaminated sub-batch by comparing the profile observed in that sub-batch with the profile expected in the absence of the contaminant, and
ii) comparing the profile obtained in i) with a reference database.

Alternatively, a method for determining the degree of purity, as defined above, is considered, characterized in that the identification of the contaminant is also carried out for each sub-lot contaminated in
i) inferring the molecular profile of the contaminant in a contaminated sub-lot by comparing the profile observed in that sub-lot with the profile expected in the absence of the contaminant and by
ii) Comparing the profile obtained in i) with those of a reference database.

One or more contaminant profiles are thus obtained for the initial seed lot, corresponding to the sum of the contaminants in each contaminated sub-lot.

The methods described above thus make it possible to carry out quality control of seed lots, on several different traits (varietal purity, specific purity, agronomic characteristics, contamination by pathogens), in a single step, and by quantifying the presence of some of the unwanted traits or contaminants. In addition, these methods allow the detailed determination of the nature of the contaminants present, due to the use of sequencing which gives precise information that can be easily used, as well as the determination of the presence of SNPs (Single Nucleotide Polymorphism) which could not be detected by other methods (probes, amplifications, DNA chips). These methods thus bring a high precision in the characterization of the tested seed lot. They are also fast and easy to implement and thus save time and reduce the costs of seed lot analysis. Thus, these methods simplify the analyses of specific purity, which are currently carried out in a tedious way by operators. They also allow the rapid testing and detection of a large number of pathogens (and also characterize their genotype according to the sequenced genes), which is currently done by potential growth of pathogens. The agronomic character of the lot (including everything related to germination and vigor) can be determined by the presence of expression of unfavorable genes, rather than by germination of seed samples, thus saving time and resources.

Thus, the methods described improve the accuracy of seed lot control, especially when they are combined.

These same methods can also be transposed and used for the study of the conformity of plants marketed in the form of seedlings, species with vegetative propagation, the evaluated material will then be made up of plant tissue samples, the quantity of which will be equivalent from one plant to another, this plant tissue could be, among others, a leaf disc.

### DESCRIPTION OF THE FIGURES

FIG. 1: Taqman analysis result for a SNP, comprising two allelic forms detected respectively by the fluorochromes FAM and VIC, in maize samples homozygous (A, B) or heterozygous for the SNP (C). A: homozygous sample for the allelic form detected in FAM. B: homozygous sample for the allelic form detected in VIC. C: heterozygous sample for the allelic forms detected in FAM and VIC.

FIG. 2: Relative frequency, in each sub-lot, of the allele alternative for SNP10. Sub-lots 3, 14 and 16 show a significant frequency of the alternative allele.

FIG. 3: Qualitative profile (presence/absence of a contaminating allele). Profile of presence of an alternative allele for the 17 markers (row) (16 discriminatory markers and one marker associated with a trait) within the 16 sub-lots (column). The presence of an alternative allele is detected for at least 3 SNPs in sub-lots 3, 14 and 16. These sub-lots are declared contaminated. The remaining 13 sub-lots are declared uncontaminated.

FIG. 4: Molecular profiles obtained on the 17 SNPs (16 discriminatory markers and one marker associated with a trait) obtained from the 16 sub-lots analyzed. The profile of the first line corresponds to the main profile, the subsequent profiles to the contaminated profiles observed for lots 3, 14 and 16 respectively.

### EXAMPLES

### Example 1: Contaminant Detection by Taqman

This example evaluates the possibility of detecting a contaminating seed in a sub-lot of maize seed, by genotyping using the Taqman (Applied Biosystem) technology.

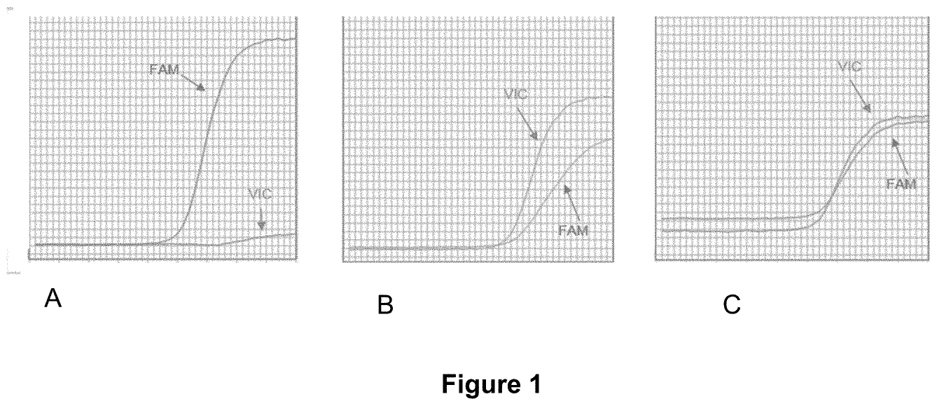

FIG. 1 shows the result of the Taqman analysis for a SNP, comprising two allelic forms detected respectively by the fluorochromes FAM and VIC, in maize samples that are homozygous or heterozygous to the SNP, and highlights the presence of a signal with the FAM probe in a sample that is homozygous for the VIC allele (B), i.e. a non-specific signal that does not distinguish a false positive signal from a signal related to real contamination in a sample.

These results show that the Taqman method does not reliably detect contaminants.

### Example 2: Detection of Contaminants by Genotyping on a Chip

In this example, batches of 200 seeds from a line A containing 10%, 20%, 30%, 40%, and up to 90% contaminants from a line B were prepared and a sample of 15 seeds from this batch was analyzed by genotyping on an Infinium (Illumina) chip, in order to assess the feasibility of identifying a contamination. Contaminations higher than 10% can be detected, but mixtures containing 10% contamination are not distinguishable from uncontaminated controls. A fortiori, the less important contaminations will not be detectable.

### Example 3: Implementation of the Method According to the Invention on a Set of Markers

In this example, a set of 16 discriminating markers (SNPs) was used, allowing the unambiguous identification of the presence of a variety other than the expected one. This set of 16 markers was defined from reference genotyping data on several thousand markers for the varieties of interest, and allows each variety to be differentiated from the others by at least 3 discriminatory markers. In this case, it is the overall molecular profile of the 16 markers that determines the identity of each variety. Each marker is specific to a locus of interest.

In an experiment under controlled contamination conditions, 24 seeds of a pure L1 line were introduced in a batch of 2376 seeds of a pure L2 line, the batch thus obtained has a 99% purity level, the seeds were randomly distributed in twenty-four sub-lots of 100 grains (i.e. 2400 analyzed grains). Each batch of seeds thus obtained was crushed independently and DNA was extracted from the crushed seeds. Thus, there is an average of 1 contaminant per batch: the number of sub-lots is indeed equal to the number of contaminants present in the complete seed batch. Due to the statistical random distribution, however, it is known that some sub-lots will not contain contaminants, and that other sub-lots will contain several contaminants, due to the sampling by forming the sub-lots.

For each of the 16 markers, an amplicon of 70 to 120 bp was defined, and the 16 markers were co-amplified by multiplex PCR. A unique index (TAG) is used for each DNA sample, allowing sequencing of all the amplicons and attribution of the sequences obtained to their original batch.

The amplicons have been sequenced by the. Illumina technology on a Miniseq sequencer. Paired sequences of 75 bases were generated, assigned to the original DNA by a demultiplexing step. After removal of adaptor sequences and of poor quality bases (Q30 threshold), each pair of sequences was reassembled into a single sequence and aligned to the reference maize genome (RefGenV4). For each SNP, the relative allele frequencies of the main and alternative allele were calculated, and correspond to the number of readings containing the allele of interest relative to the sum of the readings of each allele.

Contamination is considered to occur for an SNP marker if, in a sub-lot, the sequence of an allelic form, which is not that of the allele expected for the variety tested, appears to be greater than the background.

A sample is declared contaminated when it contains at least 3 SNPs for which an alternative allele is detected. Thus, it is concluded that, among these 24 sub-lots, 13 are considered contaminated and 11 are considered pure.

The number of contaminated sub-lots is used to estimate the varietal purity of the lot analyzed. This calculation is performed using the Seed Calc software, which uses the formulas of Remund (2001). In this example, the estimated purity is 99.22% (98.64%-99.6%), for a controlled true purity of 99%.

The estimation of the impurity \hat{p} of the batch is obtained according to the formula:

$$\hat{p} = 1 - {( 1 - \frac{d}{n} )}^{\frac{1}{m}}$$

In which n is the number of pools; m is the number of grains in a pool; d is the number of pools in which a contaminant has been identified.

In the above case: 1−(1−13/24)^{0.01}=1-0.9922=0.0078 or a purity of 99.22. The confidence interval is also calculated according to the procedures described in Remund 2001.

### Example 4: Identification of the Contaminant

In this example, basic seed lots of maize were analyzed using the same approach as in Example 3. For one lot, 16 sub-lots of 100 seeds were formed.

The seeds from each sub-lot were crushed and the DNA extracted. A set of 17 markers was identified, including 16 discriminating SNPs (allowing unambiguous identification of the presence of a variety other than the expected one) and one marker associated with a trait. For each marker, a 70-120 bp amplicon was defined, and the 17 markers were co-amplified by multiplex PCR. A unique index (Tag) is used for each DNA sample, allowing the sequencing of all the amplicons and the attribution of the sequences obtained to their original batch.

The amplicons were sequenced using Illumina technology on a Miniseq sequencer. Paired sequences of 75 bases were generated and assigned to the original DNA by a demultiplexing step. After removal of adaptor sequences and of poor quality bases (Q30 threshold), each pair of sequences was reassembled into a single sequence and aligned to the reference maize genome (RefGenV4). For each SNP, the relative allele frequencies of the main and alternative allele were calculated, and correspond to the number of readings containing the allele of interest relative to the sum of the readings of each allele.

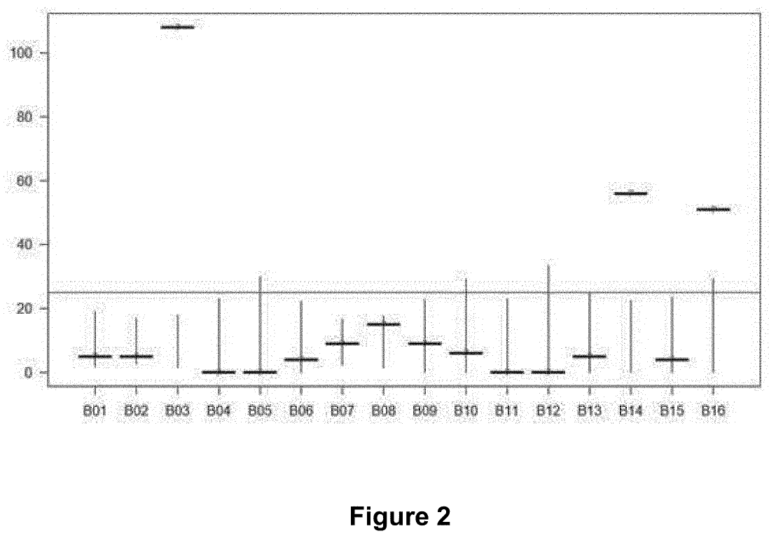

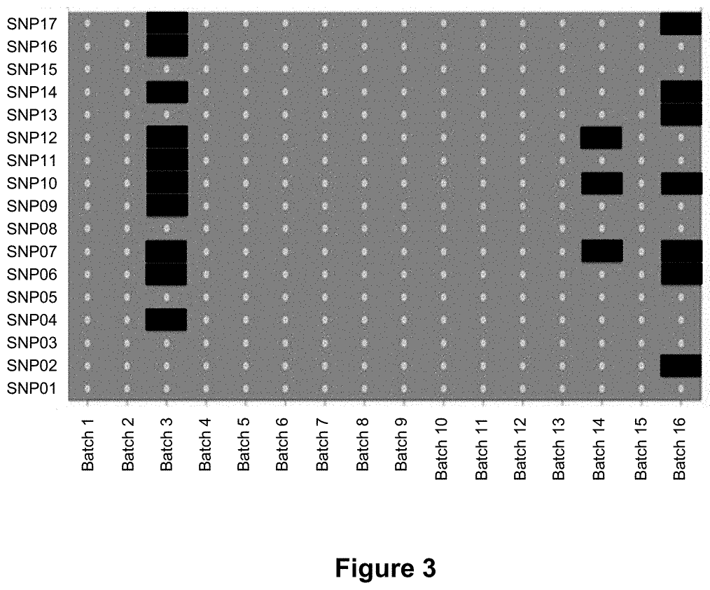

FIG. 2 shows, for an SNP (SNP10), the frequency of the alternate allele in each of the sub-lots (i.e. the frequency of occurrence of the alternate allele sequence). In this example, sub-blots 3, 14 and 16 show a significant presence of the alternate allele (above the background noise represented by the horizontal line). This analysis is performed for each SNP, and FIG. 3 shows the qualitative profile (presence/absence of the alternate allele) obtained for each SNP in each sub-lot. The presence of an alternative allele is confirmed for at least 3 SNPs in sub-lots 3, 14 and 16. These 3 sub-lots are declared contaminated. The remaining 13 sub-lots are declared uncontaminated. The varietal purity estimated with SeedCalc is 99.79% (95% confidence interval: 99.39%-99.96%).

In parallel, the same batch was analyzed on 558 individual seeds. For each seed, a fragment was taken by punching the embryo with a punch, then DNA was extracted and genotyped was performed using KASP technology (LGC Genomics) on 16 discriminatory markers. This analysis estimates a purity of 99.46% (95% confidence interval: 98.42%-99.89%).

The marker SNP17 was analyzed separately and makes it possible to estimate the purity of the associated trait.

FIG. 3 shows that sub-lots 3 and 16 show a significant frequency of the alternative allele. These 2 sub-lots are declared contaminated, leading to a line purity estimate of 99.87% (95% confidence interval: 99.52-99.98%).

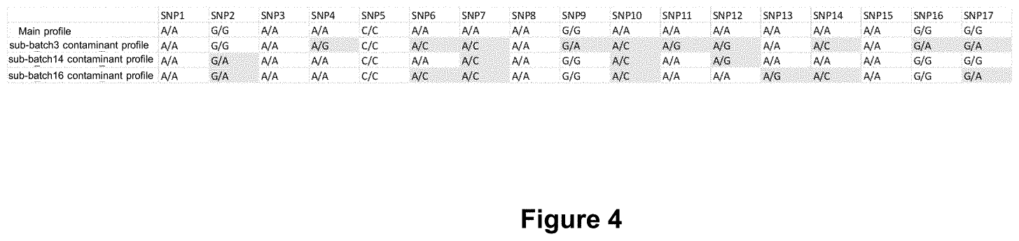

The molecular profile identified on the non-contaminated sub-lots is first used to check its conformity with the expected profile for the analyzed variety (the previous step verifies the varietal purity of the batch, this step verifies that the identified variety is indeed the expected one). Then, on sub-lots 3, 14 and 16 showing contamination, a contaminant molecular profile is deduced from the observed molecular profile, by subtraction of the expected profile. For each SNP marker showing contamination, the 2 observed alleles are reported (FIG. 4). The contaminant can thus be homozygous for the minority allele, or heterozygous.

Each contaminant molecular profile is then compared with a reference database in order to identify it. If this genotype corresponds to a known accession, it is proposed as a potential contaminant, otherwise the contaminant genotype is declared non-identifiable.

This reference database can be refined according to the production plan, in particular this database will then contain as a priority all the varieties grown in the production sector of the line. And in this context, a contaminant which will not appear in this reference database will be qualified as a contaminant related to the post-harvest process.

### Example 5: Implementation of the Method for Simultaneous Assessment of Varietal Purity and Germinative Quality of a Seed Lot

In this example, 16 sub-lots of 100 seeds are formed, so that the seed lot is evaluated on a sample of 1600 seeds. From each sub-lot, DNA and RNA are co-extracted.

For this purpose, each sub-lot is mechanically ground into a tube by adding stainless steel beads. The tubes and the grinding support are previously cooled in liquid nitrogen in order to preserve the integrity of the nucleic acids, in particular RNA. Co-extraction of DNA and RNA is performed using Macherey-Nagel's total DNA, RNA and protein isolation NucleoSpin® TriPrep kit. In a first step, a lysis buffer is added to the milled material, allowing the destruction of cell structures and the simultaneous inactivation of enzymes such as RNases. The lysates are then deposited on columns containing a silica membrane to which DNA and RNA molecules are attached. A first elution in a specific buffer elutes the DNAs while keeping the RNAs attached to the silica membrane. After a treatment with DNAse degrading DNA residues, the RNAs are washed and then eluted in RNAse free water.

For each sub-lot, a reverse transcription is performed, primed with oligo-dT oligonucleotides to synthesize double-stranded DNA complementary to the messenger RNAs present in each sample. A DNA mixture is then constituted for each sub-lot, composed of the extracted genomic DNA and the cDNAs synthesized from the RNA fraction.

A multiplex PCR is performed on each DNA sample in order to specifically amplify the targets of interest in the form of 70 to 120 bp amplicons. These amplicons correspond to the genomic regions of interest for the determination of the varietal identification molecular profile on the one hand (set of discriminant SNPs), and to the DOG1 gene, marker of the seed dormancy state on the other hand. A unique index (TAG) is used for each DNA sample, allowing sequencing of all the amplicons and attribution of the sequences obtained to their original sub-lot. Amplicons are sequenced using Illumina technology, generating paired sequences of 75 bases each. These sequences are then assigned to the original DNA by a demultiplexing step, and then undergo various treatments consisting of the removal of adaptor sequences and of poor quality bases (Q30 threshold). Each pair of sequences is finally assembled into a single sequence and aligned with the reference genome sequence.

For each SNP, the relative allele frequencies of the main and alternative alleles were calculated, and correspond to the number of readings containing the allele of interest relative to the sum of the readings of each allele. Contamination is considered to occur for an SNP marker if, in a sub-lot, the sequence of an allelic form, which is not that of the allele expected for the variety tested, appears to be greater than the background. A sample is declared contaminated when it contains at least 3 SNPs for which an alternative allele is detected. The number of contaminated sub-lots is used to estimate the varietal purity of the lot tested. This calculation is performed using the Seed Calc software which uses the formulas of Remund (2001).

With regard to the DOG1 gene, a sub-lot is considered to contain a dormant seed if specific transcript sequences of this gene are detected in an amount significantly different from the background, the expression of this gene being negligible in non-dormant seeds. This threshold of significance is previously determined using a standard range. The dormancy rate is then estimated by counting the number of sub-lots for which DOG1 gene expression is detected, using the calculation method previously used.

1. A method for determining the quantity of contaminants at at least one locus of interest, present in a seed lot of a variety of interest comprising:
a) grouping seeds from a seed lot into sub-lots of at least 10 seeds, the number of sub-lots so obtained being greater than or equal to 10,
b) performing targeted sequencing of at least the region of the seed genome containing the locus of interest for each sub-lot,
c) qualitatively determining the presence of a contaminant for each sub-lot by detection of an allele alternative to the expected allele(s) for each sequenced genomic region (presence/absence of the expected allele(s)), and
d) determining the quantity of contaminants in the overall lot by compiling the qualitative results obtained for all sub-lots.

2. The method according to claim 1, wherein the sequencing of step b) is performed on the DNA extracted from the seeds present in a sub-lot, the region of the seed genome containing the locus of interest being optionally amplified.

3. The method according to claim 1, wherein steps b), c) and d) are carried out for several regions of the genome corresponding to several loci of interest.

4. The method according to claim 3, wherein a subset of these loci of interest is sufficient to identify the variety of interest.

5. The method according to claim 4, wherein a lot is declared as containing a contaminant if an allele alternative to the expected allele(s) is observed for a single locus of interest.

6. The method according to claim 4, wherein a lot is declared as containing a contaminant if an allele alternative to the expected allele(s) is observed for more than one locus of interest.

7. The method according to claim 1, wherein at least one locus of interest is linked to a trait of interest.

8. The method according to claim 3, wherein a combination of loci is linked to characters of interest (trait).

9. The method according to claim 3, wherein a combination of loci is linked to a character of interest (trait).

10. The method according to claim 1, wherein at least one locus of interest is linked to a specific trait a priori not present in the seeds of the batch, in order to detect the fortuitous presence of this trait.

11. The method according to claim 10, wherein the lot is considered to be non-compliant if the frequency of the trait is greater than 10% in the seed lot.

12. The method according to claim 1, wherein
i) RNA is extracted from the seeds of the sub-lot and reverse transcribed into cDNA prior to step b),
ii) sequencing of this cDNA is performed using primers specific to genes related to an agronomic property of the seeds, at the same time as the sequencing of step b) is performed,
iii) the presence of seeds with the agronomic property is qualitatively determined for each sub-lot, in case of detection of cDNA relating to the specific genes of the agronomic property of the seeds in the sequencing step (ii) (presence/absence of cDNA), and
iv) the quantity of seeds with this agronomic characteristic in the overall lot is determined by compiling the qualitative results obtained for all sub-lots in (iii).

13. The method according to claim 12, wherein the agronomic property of the seeds is selected from state of dormancy, priming quality, germination ability, vigor, and viability of the seeds.

14. The method according to claim 1, wherein
i) DNA sequencing of the sub-lots is carried out using primers specific to one or more species different from those of the seeds present in the sub-lot, at the same time as the sequencing of step b) is performed,
ii) the presence of seeds of different species is determined qualitatively for each sub-lot, in case of detection of genes belonging to the said species (presence/absence of genes specific to other species), and
iii) the quantity of exogenous seeds in the overall lot is determined by compiling the qualitative results obtained for all sub-lots in ii).

15. The method according to claim 14, wherein at least one different species is a weed.

16. The method according to claim 1, wherein
i) sequencing of DNA or cDNA contained in the sub-lots using pathogen species-specific primers is carried out at the same time as the sequencing of step b) is performed,
ii) the presence or absence of DNA of the pathogenic species is determined for each sub-lot if sequences belonging to those pathogenic species are detected, or
iii) the conclusion as to the contamination of the lot is based on the presence of sequences belonging to the said pathogenic species.

17. The method according to claim 16, wherein the pathogenic species is a bacterium, a fungus, a virus, or an insect.

18. The method according to claim 1, wherein before step b)
i) DNA is extracted from each sub-lot of seeds,
ii) RNA is extracted from each seed sub-lot and reverse transcribed into cDNA,
iii) the DNA extracted in i) and the cDNA obtained in ii) are mixed,
iv) optionally, an amplification is performed on the DNA obtained in iii), specific to certain loci, or non-specific, and
v) the DNA obtained in iii) or the amplification products obtained in iv) are used as a template for the sequencing step.

19. The method according to claim 18, wherein step iv) is carried out by amplifying specific sequences of other organisms whose absence or presence is to be verified.

20. The method according to claim 18, wherein step iv) is carried out by amplifying specific sequences making it possible to determine certain agronomic properties of the seeds of the sub-lot.

21. The method according to claim 20, wherein at least one agronomic property of the seeds is selected from state of dormancy, priming quality, germination ability, vigor, and viability of the seeds.

22. The method according to claim 1, wherein the quantity of seeds in each sub-lot prepared in step a) is between 80 and 120.

23. The method according to claim 1, wherein the quantity of seeds in each sub-lot prepared in step a) is between 15 and 25.

24. The method according to claim 1, wherein the identification of the contaminant for each contaminated sub-lot is also carried out by
i) inferring the molecular profile of the contaminant in a contaminated sub-lot by comparing the profile observed in that sub-lot with the profile expected in the absence of the contaminant, and by
ii) comparing the profile obtained in i) with those of a reference database.
